(19) United States
(12) Patent Application Publication
(10) Pub. No.: US 2010/0228297 A1
(43) Pub. Date: Sep. 9, 2010

(54) SELECTIVE IMPLANTATION KIT AND METHOD INCLUDING TOOL FOR SPACER AND/OR CONTROLLED SUBSIDENCE DEVICE

(75) Inventors: Robert S. Bray, Studio City, CA (US); James M. Moran, North Royalton, OH (US); Mark T. Whiteaker, Rocky River, OH (US)

Correspondence Address: PEARNE & GORDON LLP, 1801 EAST 9TH STREET, SUITE 1200, CLEVELAND, OH, 44114-3108, US

(73) Assignee: RSB SPINE LLC, Cleveland, OH (US)

(21) Appl. No.: 12/716,726

(22) Filed: Mar. 3, 2010

Related U.S. Application Data
(60) Provisional application No. 61/156,949, filed on Mar. 3, 2009.

(51) Int. Cl. A61B 17/88; A61B 17/56

(52) U.S. Cl. 606/279; 606/86 A

(57) ABSTRACT

Kit and associated method with at least one tool for achieving selective implantation. The kit includes a fixation device for implant location intermediate the two bone bodies, a spacer for implant location intermediate the two bone bodies and adjacent the fixation device, and at least one implantation tool. The at least one tool has a portion for engaging and holding at least one of the device and the spacer during implantation of the at least one of the device and the spacer for implant location intermediate the two bone bodies and for releasing the held at least one of the device and the spacer subsequent to implantation. The at least one tool may be a single tool for engaging and holding both.

### RELATED APPLICATIONS

Benefit of priority is claimed from U.S. Provisional Patent Application No. 61/156,949, the disclosure of which is incorporated herein by reference.

### FIELD OF THE INVENTION

The present invention relates generally to selective implantation via a kit and associated method. The present invention also relates generally to the use of at least one tool for implantation of a spacer and/or a controlled subsidence device as part of the kit and associated method. Such kit and associated method for implantation of a spacer and/or a controlled subsidence device is useful for spinal surgery.

### BACKGROUND OF THE INVENTION

The spinal column of vertebrae provides support to bear weight and protection to the delicate spinal cord and spinal nerves. The spinal column includes a series of vertebrae stacked on top of each other. There are typically seven cervical (neck), twelve thoracic (chest), and five lumbar (low back) segments. Each vertebra has a cylindrical shaped vertebral body in the anterior portion of the spine with an arch of bone to the posterior, which covers the neural structures. Between each vertebral body is an intervertebral disc, a cartilaginous cushion to help absorb impact and dampen compressive forces on the spine. To the posterior the laminar arch covers the neural structures of the spinal cord and nerves for protection. At the junction of the arch and posterior vertebral body are articulations to allow movement of the spine.

Various types of problems can affect the structure and function of the spinal column. These can be based on degenerative conditions of the intervertebral disc or the articulating joints, traumatic disruption of the disc, bone or ligaments supporting the spine, tumor or infection. In addition congenital or acquired deformities can cause abnormal angulation or slippage of the spine. Slippage (spondylolisthesis) anterior of one vertebral body on another can cause compression of the spinal cord or nerves. Patients who suffer from one of more of these conditions often experience extreme and debilitating pain, and can sustain permanent neurological damage if the conditions are not treated appropriately.

One technique of treating these disorders is known as surgical arthrodesis of the spine. This can be accomplished by removing the intervertebral disc and replacing it with bone and immobilizing the spine to allow the eventual fusion or growth of the bone material across the disc space to connect the adjoining vertebral bodies together. The stabilization of the vertebra to allow fusion is often assisted by a surgically implanted device to hold the vertebral bodies in proper alignment and allow the bone to heal, much like placing a cast on a fractured bone. Such techniques have been effectively used to treat the above-described conditions and in most cases are effective at reducing the patient's pain and preventing neurological loss of function. However, there are disadvantages to the present stabilization devices.

Several types of anterior spinal fixation devices are in use currently. One technique involves placement of screws all the way through the vertebral body, called bicortical purchase. The screws are placed through a titanium plate but are not attached to the plate. This device is difficult to place, and over penetration of the screws can result in damage to the spinal cord. The screws can back out of the plate into the surrounding tissues, as they do not fix to the plate. Several newer generation devices have used a unicortical purchase of the bone, and in some fashion locking the screw to the plate to provide stability and secure the screw from back out. Problems have resulted from over rigid fixation and stress shielding, resulting in nonunion of the bony fusion, chronic micro-motion during healing resulting in stress fracture of the fixation device at either the screw or the plate, insecure locking of the screw to the plate resulting in screw back out, or inadequate fixation strength and resultant collapse of the graft and angulation of the spine.

These devices are often designed to support and bridge across a group of vertebrae, for example a group of three. Because these devices are typically bridged across the bone, for example in the cervical region, they occasionally aggravate the esophagus, making it difficult for one to swallow food. In addition, the screws are installed into the bone normal, i.e., 90° to the plate's surface. Local angularity in the vertebral column often causes high shearing stresses to be applied to the screws. These stresses may fatigue the screws or cause deformation of the screw openings.

Recent advances have provided device arrangements that are placed between the vertebrae. Such device arrangements often include a device and a spacer. Often individual surgeons have differing techniques, desires, methodologies and the like concerning fixation using such device arrangements. It would be useful to aid the surgeons with their differing techniques, desires, methodologies and the like concerning fixation.

### SUMMARY OF THE INVENTION

The following presents a simplified summary of the invention in order to provide a basic understanding of some aspects of the invention. This summary is not an extensive overview of the invention. It is intended to neither identify key or critical elements of the invention nor delineate the scope of the invention. Its sole purpose is to present some concepts of the invention in a simplified form as a prelude to the more detailed description that is presented later.

In accordance with one aspect, the present invention provides a kit for achieving selective implantation with respect to two bone bodies. The kit includes a fixation device for implant location intermediate the two bone bodies, a spacer for implant location intermediate the two bone bodies and adjacent the fixation device, and at least one implantation tool. The at least one tool has a portion for engaging and holding at least one of the device and the spacer during implantation of the at least one of the device and the spacer for implant location intermediate the two bone bodies and for releasing the held at least one of the device and the spacer subsequent to implantation.

In accordance with another aspect, the present invention provides a method for achieving selective implantation with respect to two bone bodies. The method including providing a fixation device for implant location intermediate the two bone bodies, providing a spacer for implant location intermediate the two bone bodies and adjacent the fixation device, and selecting to implant the device and the spacer together or separately as at least one of the device and the spacer. The method also includes implanting the selected at least one of the device and the spacer at an implant location intermediate the two bone bodies using at least one implantation tool. The at least one tool have a portion for engaging and holding the at least one of the device and the spacer during implantation of the at least one of the device and the spacer and for releasing the held at least one of the device and the spacer subsequent to implantation.

In accordance with another aspect, the present invention provides an implantation tool that includes a first portion for engaging and holding a fixation device during implantation at a location intermediate two bone bodies, and a second portion for engaging and holding a spacer during implantation, simultaneous with the implantation of the fixation device, at the location intermediate the two bone bodies and adjacent to the fixation device. The first and second portions are configured to release the held device and spacer subsequent to implantation.

In accordance with still another aspects, the present invention provides that the kit, the method and the tool are for achieving selective implantation of the device as a controlled subsidence device. The bone bodies have relative superior and inferior locations. The device includes first and second lateral portions for location on first and second lateral sides of a chamber to be at least partially bounded by the device and the bone bodies, and at least one interface member extending transverse to a lateral direction and configured to provide controlled subsidence of the at least one interface member into at least one of the bone bodies. The spacer and the device are relatively sized such that the lateral portions of the device substantially bound the spacer in the lateral directions, and such that the spacer does not prevent controlled subsidence of the at least one interface member into the at least one of the bone bodies.

### BRIEF DESCRIPTION OF THE DRAWINGS

The foregoing and other features and advantages of the present invention will become apparent to those skilled in the art to which the present invention relates upon reading the following description with reference to the accompanying drawings.

FIG. 1 is a perspective view of an example fixation device and an example spacer implanted between two vertebrae of a spine as example bone bodies, with the device and the spacer being part of an example kit in accordance with an aspect of the present invention;

FIG. 2 is reduced, plan view of an example kit in accordance with an aspect of the present invention and that includes the device and the spacer shown in FIG. 1;

FIG. 3 is an enlarged, perspective view of a first example insertion tool and the spacer of the kit shown in FIG. 2;

FIG. 4 is reverse perspective view of the tool and the spacer shown in FIG. 3;

FIG. 5 is an enlarged view along line 5-5 in FIG. 3 and shows example details of an engagement and holding portion of the tool shown in FIG. 3;

FIG. 6 is an enlarged view along line 6-6 in FIG. 4 and shows example details of a portion of the spacer shown in FIG. 4 that can be engaged and held;

FIG. 7 is an enlarged, perspective view of a second example insertion tool and the device of the kit shown in FIG. 2;

FIG. 8 is reverse perspective view of the tool and the device shown in FIG. 3;

FIG. 9 is an enlarged view along line 9-9 in FIG. 7 and shows example details of an engagement and holding portion of the tool shown in FIG. 3;

FIG. 10 is an enlarged view along line 10-10 in FIG. 8 and shows example details of a portion of the device shown in FIG. 10 that can be engaged and held;

FIG. 11 is an enlarged, side view of the engagement and holding portion shown in FIG. 9, an enlarged, torn-off segment of the device that threadingly engages the engagement and holding portion shown and an enlarged, torn-off segment of the spacer that wedgingly engages the engagement and holding portion shown;

FIG. 12 is top level flow chart for an example method in accordance with an aspect of the present invention;

FIG. 13 is an enlarged, perspective view of a portion of a tool and a fixation device that may be part of another example kit; and

FIG. 14 is a reverse angle perspective of the tool portion and device of FIG. 13 and also another example spacer.

### DESCRIPTION OF EXAMPLE EMBODIMENTS

The present invention will now be described with reference to the drawings, wherein like reference numerals are used to refer to similar elements throughout. It is also to be appreciated that the various drawings are not necessarily drawn to scale from one figure to another nor inside a given figure, and in particular that the size of the components are arbitrarily drawn for facilitating the understanding of the drawings. Additionally, other embodiments of the invention are possible and the invention is capable of being practiced and carried out in ways other than as described. The terminology and phraseology used in describing the invention is employed for the purpose of promoting an understanding of the invention and should not be taken as limiting.

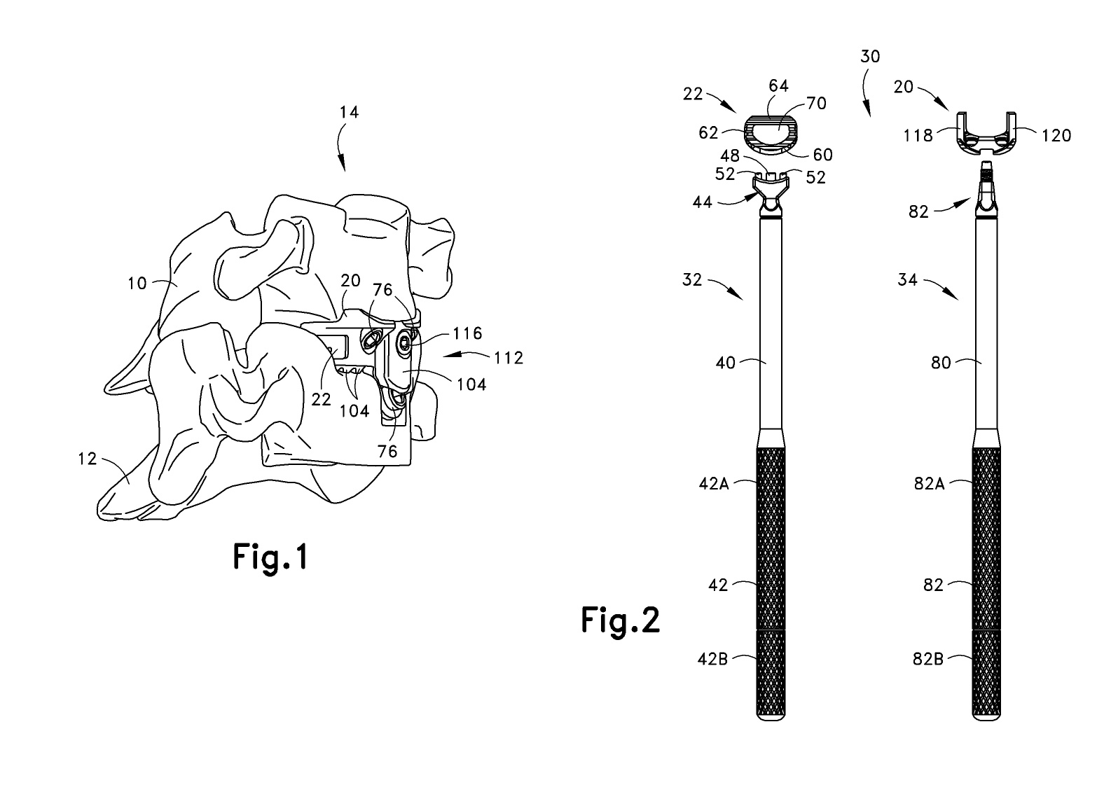

For one example environment within which the present invention may be utilized, attention is directed to the field of surgically implanted devices between two bone bodies. As used herein, the phrase “bone bodies” is intended to include individual bones as well as fragments or portions of bones. In a specific example environment, the bone bodies are adjacent vertebrae 10, 12 (e.g., spinal members) of a spine 14. See FIG. 1, which shows that the upper vertebra 10 is at a relative superior location and the lower vertebra 12 is at a relative inferior position. The two vertebrae 10, 12 have respective opposing surfaces directed toward each other, which are thus superior and inferior surfaces, respectively. As a still further specific example, the spine 14 is a mammalian spine and could be a human spine.

Often, a device arrangement (e.g., 18) is implanted in an implant location that is a space volume intermediate (e.g., between) two such adjacent vertebrae 10, 12. Specifically, the device arrangement 18 would be located within the space volume between the two vertebrae 10, 12 and thus has superior and inferior surfaces facing the two vertebrae. Thus, such location between the two vertebrae 10, 12 is an example selective implant location and FIG. 1 shows such an example. Such an implanted device arrangement 18 may include a fixation device 20 within the space volume intermediate the two vertebrae 10, 12. The fixation device 20 is for fixing and securing. In the specific shown example the fixation device 20 for controlled subsidence. Also, such an implanted device arrangement 18 may include a spacer 22 that can be located intermediate the two vertebrae 10, 12 with the fixation device 20. Thus, the spacer 22 is adjacent to the fixation device 20 within the space volume between the two vertebrae 10, 12. In the shown example, the spacer 20 is located a chamber partially defined by the device 20.

At the outset, it should be appreciated that in the shown example the device 20 and the spacer 22 are separate parts. The fixation device 20 and the spacer 22 are not attached to each other. Also, it is to be appreciated that although the shown example fixation device 20 is for controlled subsidence it is fully within the present invention that the fixation device can be a device that is not for controlled subsidence. As such, it is to be appreciated that the fixation device 20 can be significantly different from the shown example and that such different constructions, configurations, etc. are within the present invention. Similarly, the spacer 22 may differ significantly from the shown example and that such different spacer constructions, configurations, etc. are within the present invention. It is possible that differences in the construction, configuration, etc. for the spacer may be guided by the construction, configuration, etc. of the fixation device 20.

Focusing upon the example of the fixation device 20 being for controlled subsidence, it is to be appreciated that for even such a beneficial feature, the shown device 20 and the shown spacer 22 are merely examples. It is to be appreciated that the construction, configuration, etc. of the device 20 and/or spacer 22 may be varied. A few examples of variations of the device 20 are set forth in U.S. Pat. No. 6,984,234 and U.S. Published Application No. 2006/0030851, which are incorporated herein by reference. As will be appreciated, the example controlled subsidence device 20 can fix and secure the adjacent vertebrae 10, 12 that have had a cartilaginous disc between the vertebrae removed to create the volume for receiving the device 20 and spacer 22. Although the device 20 is for fixing and securing, controlled subsidence (e.g., movement) can occur such that stress shielding is avoided and load sharing occurs.

Typically, such implantation is associated with the removal of all or at least part of the disc located between the vertebrae (e.g., a discectomy). It is possible that a surgeon conducting such a procedure would select (i.e., chose) to implant the spacer 22 separately from implantation of the associated controlled subsidence device 20. With regard to separate implantation, the spacer 22 may be implanted prior to implantation of the device 20. Such separate implantation of the spacer 22 may allow for the spacer to help maintain vertebral spacing between the vertebrae 10, 12 while other procedures are accomplished prior to implantation of the associated controlled subsidence device 20. Examples of such other procedures include further cleaning of the space between the vertebrae 10, 12. Also, it is possible that a surgeon conducting such a procedure would select (i.e., chose) to implant the spacer 22 together with the associated controlled subsidence device 20. Such simultaneous implantation may allow for shorter surgery time. A kit 30 (see the example in FIG. 2) and associated method in accordance with aspects of the present invention provide for such choices and benefits. Of course, other benefits are possible when the surgeon is provided with the options provided by the kit 30 and associated method in accordance with the present invention.

The kit 30 shown in FIG. 2 is an example in accordance with such an aspect of the present invention. The example kit 30 includes the example controlled subsidence device 20 for implantation at a location intermediate (e.g., between) two adjacent bone bodies (e.g., the vertebrae 10, 12), the example spacer 22 for implantation also at the location intermediate the two bone bodies (e.g., the vertebrae 10, 12), and at least one implantation tool (e.g., 32 or 34) that has a portion for engaging and holding at least one of the device and the spacer during implantation of the at least one of the device and the spacer for implant location intermediate the two bone bodies and for releasing the held at least one of the device and the spacer subsequent to implantation. Within the shown example, the kit 30 includes two tools (i.e., a first tool 32 and a second tool 34). It is to be appreciated that the terms “first” and “second” are merely used for convenience and ease of reference.

Within FIG. 2 it is to be noted that the first tool 32 is shown in close proximity with the spacer 22 and the second tool 34 is shown in close proximity to the device 20. As a preliminary indication, the first implantation tool 32 may be used to implant the spacer 22, alone, and the second, different tool 34 may be used to implant the device 20, alone. However, the second tool 34 may be used for simultaneous implantation (i.e., implantation of both together at the same time) of the device 20 and spacer 22 together between the two bone bodies (e.g., vertebrae 10, 12). So, in accordance with one aspect of the present invention, the second tool 34 is an instrument capable of simultaneously implanting the device and the spacer.

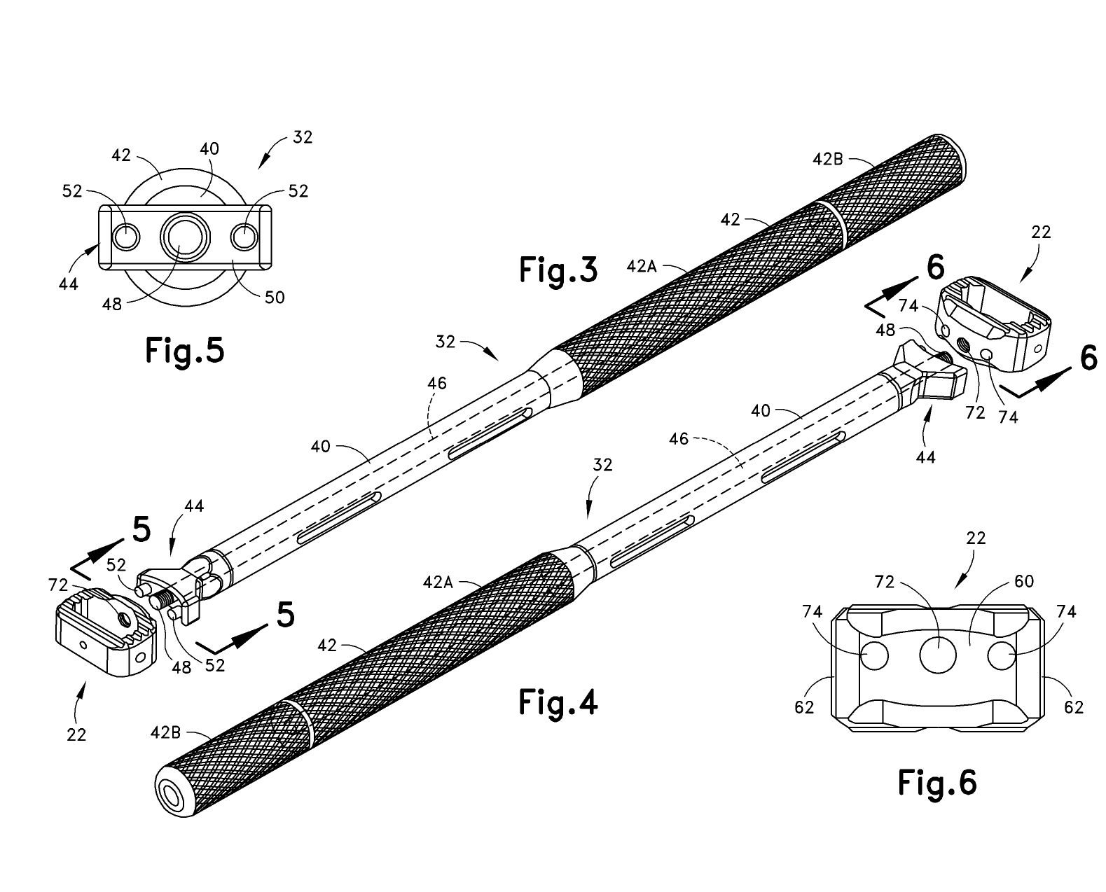

Turning to the first tool 32 (FIGS. 3 and 4), the first tool has an elongate shaft 40 with a handle end portion 42 and an engagement end portion 44. The shaft 40 is hollow. A first segment 42A of the handle end portion 42 is static and a second segment 42B of the handle end portion is relatively rotatable. Both segments 42A, 42B of the handle end portion 42 may have serration texturing or similar to provide for gripping by a surgeon (e.g., tool user). The second, relatively rotatable handle segment 42B is connected for rotation with a rod 46 (shown in phantom in FIGS. 3 and 4) that extends within the hollow shaft 40. Upon rotation of the second handle segment 42B, the rod 46 rotates within the hollow shaft 40.

At the distal end of the rod 46, the rod extends out from the shaft 40 at the engagement end portion 44. An exposed end 48 of the rod 46 is threaded. So as the rod 46 rotates, the threaded end 48 rotates. The threaded end 48 is basically centered on the shaft 40 of the tool 32 as can be seen in FIG. 5. As will be described further, the threaded end 48 may be engaged with the spacer 22. As such, the threaded end 48 is at least one aspect for engaging and holding the spacer 22. The specifics of the threaded end 48 (e.g., thread length, diameter, pitch, etc.) may be varied so long as threaded engagement with the spacer 22 is accomplished. It is to be appreciated that engagement and holding may be accomplished by means other than threaded engagement.

It is possible to support the rod 46, with its threaded end 48, relative to the non-rotating or static portions (e.g., the shaft 40 and the engagement end portion 44) via various means. For example, bearing surfaces, bushings, lubricants, seals, etc. may be utilized. Still further, one or more structures to permit some axial (i.e., elongate dimension of the tool shaft) movement of the rod 46 relative to the static portions (e.g., shaft 40) and/or prevention of axial movement may be incorporated into the tool 32.

Also, at the engagement end portion 44 the first tool 32 has non-rotating structure for engaging the spacer 22. In the shown example, the engagement end portion 44 is a head with an engagement face 50 (see FIG. 5) that is at least partially contoured to match/mate with the spacer 22. In the shown example, the engagement face 50 has a slight concave, generally cylinder contour (see FIGS. 3 and 4). Also in the shown example, the engagement end portion 44 (see FIGS. 3 and 5) includes two non-threaded protrusions 52 on the engagement face 50 and located on respective sides of the threaded end 48. In the shown example, the two protrusions 52 are cylinder shaped with round cross-sections, however, the projections may have a varied shape or may be otherwise varied.

The portions of the first insertion tool 32 can be made from any suitable material, such as metal, stainless steel, titanium, alloys such as titanium alloy, and the like. Further, the portions of the first insertion tool can be sterilized, passivated and electropolished as known in the art. The surfaces of the components, other than the gripping textures on the handle end portion, are burr-free to allow for a smooth surface that moves easily against a patient's skin and internal tissue.

Focusing upon the spacer 22, it is to be appreciated the spacer may have a varied construction and configuration and the shown spacer is only one example. With this understanding, some details of the shown example are provided. The spacer 22 may be made of metal (e.g., titanium), plastic, polymer, bone, etc. Also, the spacer may be rigid, resilient, etc. As such, material selection is generally not a limitation upon the spacer 22. The spacer 22 has an anterior wall 60, lateral walls 62, and a posterior wall 64 and includes an anterior face, a posterior face, a superior face (upward-facing toward vertebra 10 when inserted between the vertebrae 10, 12), an inferior face (downward-facing toward vertebra 12 when inserted between the vertebrae) and lateral faces. The spacer 22 includes a hollow interior 70 that extends from the superior face to the inferior face and may be configured to house bone-growth inducing material, graft material or the like. The inner surfaces defining the hollow interior are shaped to substantially follow the outermost curved contour of the spacer 22 being arcuate except opposite the posterior face.

In one example, the side profile of the spacer 22 tapers to narrow from the anterior face to the posterior face. The superior and inferior faces have undulations formed by crests that alternate and extend transversely across the superior and anterior faces in a parallel manner. The troughs are concavely curved while the crests are configured to contact the vertebrae and the inclination of which substantially conforms to the tapering of the side profile. Each of the lateral faces, to which the anterior face transitions, includes a non-threaded opening which allows vascularities to spread to the hollow interior of the spacer 22. The openings are about 1.5 mm in diameter.

The spacer 22 is generally sized such that controlled subsidence of the device 20 is permitted to occur. Basically, the spacer 22 and the device 20 are relatively sized such that lateral portions of the device 20 substantially bound the spacer in the lateral directions, and such that the spacer does not prevent controlled subsidence of at least one interface member 104 into the at least one of the vertebrae (e.g., 12). However, the spacer 22 can be a load supporting structure when implanted between the two vertebrae 10, 12.

The anterior wall 60 of the spacer 22 includes a threaded opening 72 (see FIGS. 3, 4 and 6) for engagement by the threaded end 48 of the first insertion tool 32 and two non-threaded openings 74 (See FIGS. 4 and 6) formed symmetrically about the threaded opening 72. The two non-threaded protrusions 52 of the tool 32 mate into the non-threaded openings 74 of the spacer 22. When viewed from the top (see FIG. 2), the anterior face is arcuate in a convex manner and transitions to the lateral faces at its ends. The anterior face has a narrower portion that is bounded by two anterior angled faces the curved configuration of which is adapted to accommodate bone screws 76 (see FIG. 1) that are inserted at an angle through the corresponding device 20. Each of the lateral faces is arcuate transitioning from the anterior face to the posterior face in a smooth fashion. The posterior face is the only part of the contour of the spacer 22 that includes a flat surface. The spacer 22 includes chamfered surfaces where the posterior face and the lateral faces transition to the superior face and the inferior face such that the spacer becomes tapered toward the planar tops. The chamfering is curved in accordance with the curvature of the outermost contour of the spacer 22.

As mentioned, the spacer 22 may be configured to be used in combination with the device 20 (FIG. 1). The side profile of the spacer 22 and the device 20 are such that their heights taper equally from the anterior side to the posterior side when combined together. However, the spacer 22 and the device 20 need not interlock in the combined state and may be free to move after they are surgically inserted in between the vertebrae 10, 12.

It is to be understood that other embodiments of the spacer are contemplated. One possible embodiment of spacer is a lumbar version, which includes a transversal beam extending across the hollow interior from the anterior side to the posterior side. In this embodiment, the presence of the transversal beam allows the dimensions of the anterior wall, lateral walls and the posterior walls to be thinner than the first embodiment discussed above, which may be considered a the cervical version.

It is to be appreciated that in the shown example, the second segment 42B of the handle is rotatable to cause rotation of the threaded end 48 to tighten or loosen the threaded end within the threaded opening 72. With the threaded end 48 of the tool 32 tightened into the threaded opening 72, the spacer 22 is engaged and held by the threaded end 48. The protrusions 52 can also cooperate to engage and hold the spacer 22. Also, the protrusions 52 help prevent rotation of the spacer 22 as the threaded end 48 is rotated (i.e., threaded) into the spacer 22. Of course, rotation of the handle segment 42B, rod 46 and threaded end 48 in the opposite direction will remove the threaded end 48 from the spacer 22. As such, the spacer 22 is released and is no longer held. Such release can be done subsequent to the spacer 22 being implanted.

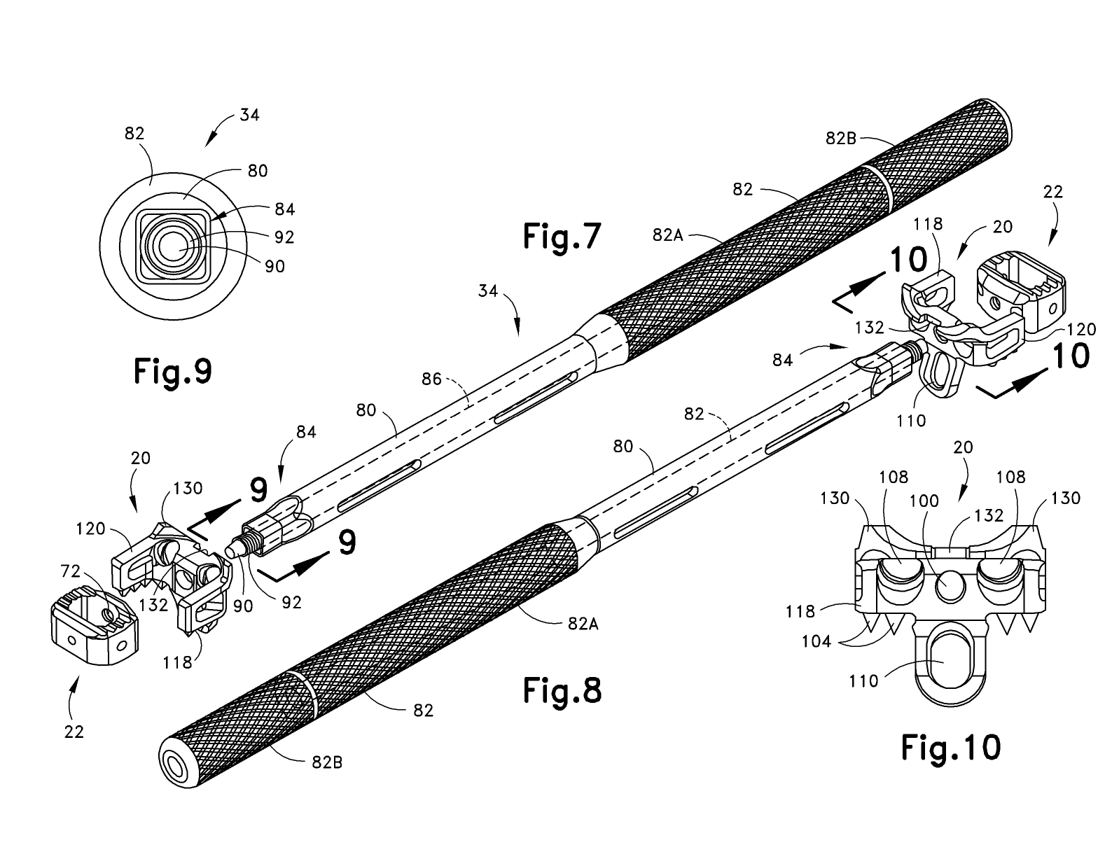

Turning to the example of the second tool 34 (FIGS. 7-9), the tool is for engaging and holding the device 20 (see FIGS. 2 and 10), alone, or the device 20 and the spacer 22 together (see FIGS. 7 and 8), during implantation of the device or device/spacer intermediate (i.e., between) the two vertebrae (bone bodies) 10, 12. The second tool 34 (FIGS. 7-9) can even hold the spacer 22 along if so desired. The tool 34 can then release the held at least one of the device 20 and the spacer 22 subsequent to implantation.

The second tool 34 has some structures that are similar to the structures of the first tool 32. For example, the second tool 34 has an elongate shaft 80 with a handle end portion 82 and an engagement end portion 84. The shaft 80 is hollow. A first segment 82A of the handle end portion 82 is static and the second segment 82B of the handle end portion is relatively rotatable. Both segments 82A, 82B of the handle end portion 82 may have serration texturing or similar to provide for gripping by a surgeon. The second, relatively rotatable handle segment 82B is connected for rotation with a rod 86 that extends within the hollow shaft 80. Upon rotation of the second handle segment 82B, the rod 86 rotates within the hollow shaft 80.

It is possible to support the rod 86 relative to the non-rotating portions of the tool 34 via various means. For example, bearing surfaces, bushings, lubricants, seals, etc. may be utilized. Still further, one or more structures to permit some axial (i.e., elongate dimension of the tool shaft) movement of the rod 86 relative to the static portion of the shaft and/or prevention of axial movement may be incorporated into the tool.

The portions of the second insertion tool 34 can be made from any suitable material, such as metal, stainless steel, titanium, alloys such as titanium alloy, and the like. Further, the portions of the insertion tool 34 can be sterilized, passivated and electropolished as known in the art. The surfaces of the portions, other than the gripping textures described below, are burr-free to allow for a smooth surface that moves easily against a patient's skin and internal tissue.

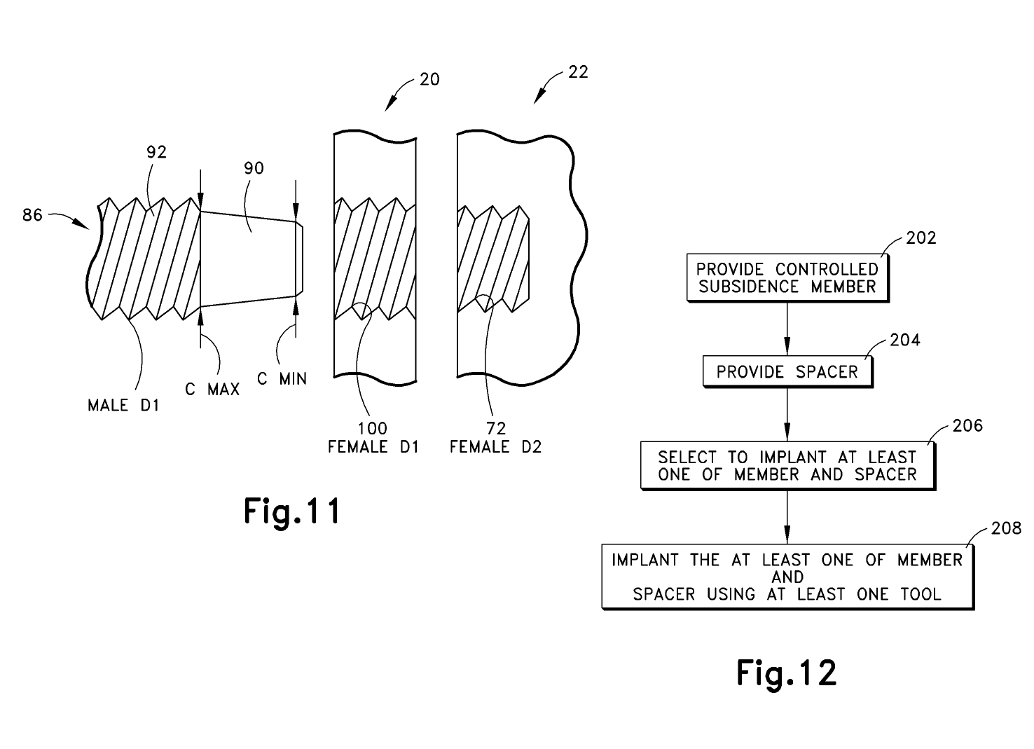

The main difference of the second tool 34 from the first tool 32 is that the engagement end portion 84 of the second tool 34 differs from the engagement end portion 44 of the first tool 32. The engagement end portion 84 of the second tool 34 is for engaging/holding holding the device 20 and possibly the spacer 22 if so desired. Specifically, at the distal end of the rod 86, the rod extends out from the shaft at the engagement end portion 84. The end portion of the rod 86 has a frustum-shaped truncated cone 90 at a tip portion of the rod, with the cone having a varying diameter circular cross-section (See FIG. 11). The cone shape has a minimum diameter of CMIN at the very tip and a maximum diameter CMAX. Herein, the frustum-shaped truncated cone 90 is referred to simply as the cone.

Axially adjacent to the cone 90 is a threaded segment 92 of the rod. The threads of the threaded segment 92 are sized (see FEMALE D1 in FIG. 11) to mate with threads of a threaded opening 100 within the device 20 (see FIGS. 10 and 11). As the rod 86 rotates, the threaded segment 92 rotates. The threaded segment 92 is basically centered on the tool 34 as can be seen in FIG. 9. As will be described following, the threaded segment 92 may be engaged with the device 20. As such, the threaded segment 92 is at least one aspect for engaging and holding the device 20. The specifics of the threaded segment 92 (e.g., thread length, diameter, pitch, etc.) may be varied so long as the engagement with the device 20 is accomplished.

Turning back to the cone 90, the cone is sized to wedge into the threaded opening 72 of the spacer 22. Specifically, the minimum diameter of the cone CMIN is smaller that the smallest diameter (see FEMALE D2 shown in FIG. 11) of the threaded opening. As such, at least some portion of the cone 90 can freely pass into the threaded opening 72 of the spacer 22. However, the maximum diameter CMAX of the cone 90 is greater than the minimum diameter FEMALE D2) of the threaded opening 72 of the spacer 22. As such, at least some portion of the cone 90 cannot enter the threaded opening 72. Due to the taper of the cone 90, the cone surface will wedge against the threads in the threaded opening 72 at some point along the cone. As such, the cone 90 is at least one aspect for engaging and holding the spacer 22.

It is to be noted that the cone 90 is sized to pass through the threaded opening 100 of the device 20. Specifically, the maximum diameter CMAX of the cone 90 is less that the minimum diameter Female D1 of the threads of the threaded opening 100 on the device 20. Thus, the rod end can reach through the threaded opening 100 on the device 20 to the spacer 22. Thus, the rod end, with the threaded segment 92 and the cone 90, is at least one aspect for engaging and holding both the device 20 and the spacer 22. The use of a cone 90 for wedging allows the threaded segment 92 of the rod end to be rotated into tight threaded engagement with the female threaded opening 100 in the device 20 without conflicting with a threading inter-engagement between the rod end and the spacer 22 since there is no threading inter-engagement between the rod end and the spacer when the second tool 34 is utilized to engage and hold both the device 20 and the spacer 22. Of course, the second tool 34 can be used to engage and hold both the device 20 and the spacer 22 or just the device 20 alone or just the spacer alone. Also, it is to be appreciated that engagement and holding may be accomplished by means other than threaded engagement and/or cone wedging.

Rotation of the handle segment 82B and rod 86, and thus threaded segment 92, in the opposite direction will remove the threaded segment 92 from the device 20. As such, the device 20 is released and is no longer held. Such release is done when the device 20 has been implanted. Such rotation in the opposite direction can also be used to remove, via a progressive pushing force, the spacer 22 from the cone 90. As such, the spacer 22 would be released and no longer held. In general, if the spacer 22 is engaged and held onto the cone 90 of the second tool 34 via wedging into the threaded opening 72 of the spacer, the spacer may be released via un-wedging from the cone. Such un-wedging can be used if only the spacer 22 is on the second tool 34.

Focusing upon the device 20 (see FIGS. 1, 7, 8 and 10), it is to be appreciated the device may have a varied construction and configuration and the shown device is only one example. With this understanding, some details of the shown example are provided. The example device 20 has a plurality of protrusions or interface members 104 extending there from. As will be explained in further detail below, the interface members 104 are configured to contact at least one surface of at least one bone body (e.g., vertebrae 10, 12) to provide subsidence control for the device 20. Controlled subsidence relates to resistance to subsidence and total amount of subsidence. The device 20 also includes a plurality of openings (two circular and one elongate to be a slot) 108 and 110, each of which is configured to receive a corresponding screw 76 there through. The bone screws 76 extend into the bone of the vertebrae 10, 12 and as such the device 20 is attached to at least one (two as shown) of the vertebrae 10, 12.

The device 20 also includes a restraining means 112 (see FIG. 1) for restricting movement of the one or more screws 76 extending there through. Such movement may include backing-out and/or pivoting during controlled subsidence and/or sliding during controlled subsidence. The restraining means 112 can be any means for securely covering at least a part of each of the screws 76 so that the screws cannot back out from the vertebrae 10, 12 once screwed in to the bone mass through the device 20. In the shown example, the bone screw restraining means 112 comprises a restraining plate 114 and a restraining plate fixing means 116.

Specifically, the fixing means 116 is a screw (e.g., a set screw) that passes through a non-threaded opening in the plate 114 and extends in threading engagement with the threaded opening 100 (FIG. 10) in the device 20. Thus, the threaded opening 100 can serve two functions. The first function being for threading engagement with the second tool 34 for holding the device 20 during insertion and the second function being for threading engagement with the screw 116 for holding the restraining plate 114.

The device 20 is generally U-shaped with a first end at the open end of the U-shape and a second end at the closed end of the U-shape (see FIGS. 7 and 8). First and second lateral side legs 118, 120 form the U-shape of the device 20. In use, the first and second legs 118, 120 extend adjacent to the lateral walls 62 of the spacer 22. The first and second legs 118, 120 are first and second lateral portions of the device 20 that are located on first and second lateral sides, respectively, of the volume between the two vertebrae 10, 12. Accordingly, the first and second legs 118 and 120 and the two vertebrae 10, 12 bound a chamber within which the spacer 22 is located. Of course, other materials may also be provided/present within the chamber bounded by the two legs 118, 120. For example, bone graft material may be present. In general, the device 20 provides stability and prevents expulsion of the spacer 22. On a more specific aspect, the legs 118, 120 help mitigate lateral shift of the spacer 22 and/or other material (e.g., graft material) during controlled subsidence and subsequent to such subsidence. The device has an angled, downwardly extending portion and one opening 110 is elongate to be a slot and extends though this portion. Relative movement of the respective bone screw 76 along the elongate direction of the slot opening 110 may be part of the subsidence movement.

Subsidence can be further controlled by the presence of at least one interface member 104. Such interface member 104 extends transverse to a lateral direction from at least one of the superior and inferior surfaces and configured to provide controlled subsidence of the at least one interface member into at least one of the vertebrae (e.g., 12). The interface members 104, as depicted in the example embodiment, can include a plurality of teeth extending from bottom surfaces of the first leg 118 and the second leg 120. Accordingly, when coupled with the bone bodies (e.g., vertebrae 10, 12), the interface members 104 extend in a direction that is aligned with an elongate direction of the spine. The interface members 104 thus, are configured to provide a progressive penetration into the bone body (e.g., vertebra 12) over a period of time in a direction aligned with the elongate direction of the spine. It is to be appreciated, however, that any suitable configuration of interface members can be provided at any suitable that interfaces with a surface of the vertebrae 10 or 12. For example, interface members could additionally or alternatively be located on the top surfaces of the first and second legs 118 and 120.

The interface members 104 can include teeth, knife-edges, spikes, posts, pegs, and the like, including any combination thereof. In the shown example, the interface members are symmetrical about two parallel axes. Specifically, the interface members 104 are not merely sloped so to be in a saw-tooth configuration. Also, the interface members 104 are configured, (e.g., sufficiently large, sufficiently spaced, etc.) so as to penetrate into the vertebrae during controlled subsidence. Thus, the interface members 104 do not merely rest upon the vertebrae surfaces for the purpose of resisting back-out of the device from the volume space between the vertebrae 10, 12.

The configuration of the interface members 104 includes interlocking external features that impact a subsidence profile, which is a relationship between an applied load and an amount of settling the device 20 experiences when secured to the vertebrae 10, 12. Or in other words, the subsidence profile is a relationship between a depth of subsidence of the interface members and a force required to achieve the depth of subsidence. When first implanted, the interface members 104 will rest on top of the bone surface. When load is applied to the device 20, the interface members 104 will penetrate, or subside, into the bone(s) in a controlled manner. The interface members 104 can penetrate into the bone “fast” initially and then slow down as more of the cross section embeds. Different interface member configurations provide different controlled subsidence profiles. The density of the bone body (e.g., vertebrae 10, 12) also impacts the subsidence profile. Still further, the amount of force needed for displacement and the rate of penetration of the interface members into the bone body (e.g., vertebrae 10, 12) depends, in part, upon the configuration of the interface members 104.

The height of the interface members 104 determines a depth of penetration into the bone body (e.g., vertebrae 10, 12). Generally, when the device 20 has subsided to a point where the interface members 104 are fully embedded in the bone, the applied load will be distributed across the entire surface of the device 20 and subsidence will cease. Typically, the screw 76 will be at the end of the slot opening 110. Thus, the height of the interface members can control an amount of subsidence that the device 20 will permit.

In addition to the height of the interface members 104, the shape of the interface members 104 also affects subsidence of the device 20. The shape of the interface members 104 controls a shape of the subsidence profile; and therefore, affects the load shared with the graft material. For instance, if the interface members 104 were limited to a few sharply pointed spikes, subsidence would occur substantially immediately and the device 20 would rapidly seat in the bone to the fullest extent under low force. In this instance, any graft material would be immediately and highly loaded. Such immediate subsidence is not desirable because the joint space could narrow and cause nerve root or spinal cord compression. Also, the graft would be overloaded, inhibiting fusion. However, some subsidence is needed to load the graft and ensure fusion. Accordingly, by configuring the interface members 104 to have a broadly shaped portion, the device 20 has increased resistance to subsidence as the interface members 104 penetrate into the bone body; and the graft material is gradually loaded as the device subsides. The substantially broad base of each interface member 104 facilitates controlled subsidence of the device 20. For instance, as the tooth shape of the interface member 104 becomes wider in cross section, the penetration of the tooth into the bone body will become slower.

Once the interface members 104 have fully penetrated the bone, the surface area of the device 20 is of an area large enough to resist further subsidence of the device 20. To increase subsidence resistance, a shelf-like area on the device 20 is provided. The shelf-like area provides an extended surface area to contact the bone material, thereby increasing subsidence resistance once the interface members 104 have fully subsided. As mentioned, the associated screw 76 will typically be at the end of the slot opening 110.

Focusing upon the slot opening 110, the slot is located in a portion extending out at an angle relative to the general U-shape of the device 20. The angle is generally from 110° to about 160°. This portion containing the slot opening 110 may extend slight outside of the volume bounded by the two adjacent vertebrae 10, 12 and may also be located is an area removed of bone material from one of the vertebra (e.g., 12) as shown in FIG. 1.

At least one and preferably two projections 130 extend upwardly from an upper portion of the device 20. The projections 130 contact a surface of the vertebrae 10, 12 to provide a stop when inserting the device 20 between the vertebrae 10, 12. Also, it is to be noted that the device 20 and its projections 130 may be configured to engage and mate with the static, non-rotating portion of the shaft 80 of the tool 34. Such engagement/mating is such that the device 20 is held against rotation as the rod 86 with the threaded segment 92 is rotated within the threaded opening 100 of the device 20. In the shown example, the shaft 80 has a generally square cross-section at the end portion of the shaft. As such in the shown example of the device 20, the space 132 between the two projections 130 is configured (e.g., generally square profile) to receive the square end of the shaft 80 such that with the square end so received, the device 20 is held against rotation relative to the shaft of the tool. Thus the rod 86 can be rotated and the threaded segment 92 can be rotationally tightened or loosened within the threaded opening 100 in the device 20. The square end of the shaft 80 may be considered a portion for engaging and holding the device 20 during implantation and for release of the held device subsequent to implantation.

In the shown example, the device 20 also includes optional lateral openings provided through each of the first and second legs 118, 120. The openings facilitate visualization of the fusion mass on x-rays and bone growth there through. The device 20 may be made of any suitable material, such as titanium or a titanium alloy or even PEEK. The thickness of the device 20 is not critical, and preferably ranges from about 1 mm to about 2 mm, and more preferably is about 1.6 mm. The height of the device 20 will depend on the needs of the particular patient.

Once the device 20 is implanted, either with or without the spacer 22, the at least one (e.g., three) bone screws 76 are positioned to extend through openings in the device 20 and into the vertebral bone material. The device 20 is configured to cooperate with the bone screws 76 to permit controlled subsidence. Specifically, one or more interface members 104 on the device 20 progress in penetration into one or both of the vertebrae. The bone screws 76 and the device 20 are configured such that the bone screws may move (e.g., slide and/or toggle) relative to the device such that the controlled subsidence is permitted.

So in general, one aspect of the present invention is a kit for achieving selective implantation with respect to two bone bodies. The kit includes a fixation device for implant location intermediate the two bone bodies, a spacer for implant location intermediate the two bone bodies and adjacent the fixation device, and at least one implantation tool. The at least one tool has a portion for engaging and holding at least one of the device and the spacer during implantation of the at least one of the device and the spacer for implant location intermediate the two bone bodies and for releasing the held at least one of the device and the spacer subsequent to implantation.

The present invention also has another specific aspect if the device is the shown example and is for controlled subsidence. Specifically, the other aspect is a kit for achieving selective implantation with respect to two bone bodies, with the bone bodies having relative superior and inferior locations. The kit includes a controlled subsidence device for implant location intermediate the two bone bodies. The device includes first and second lateral portions for location on first and second lateral sides of a chamber to be at least partially bounded by the device and the bone bodies, and at least one interface member extending transverse to a lateral direction and configured to provide controlled subsidence of the at least one interface member into at least one of the bone bodies. The kit includes a spacer for implant location intermediate the two bone bodies and within the chamber. The spacer and the device are relatively sized such that the lateral portions of the device substantially bound the spacer in the lateral directions, and such that the spacer does not prevent controlled subsidence of the at least one interface member into the at least one of the bone bodies. The kit includes at least one implantation tool. The at least one tool has a portion for engaging and holding at least one of the device and the spacer during implantation of the at least one of the device and the spacer for implant location intermediate the two bone bodies and for releasing the held at least one of the device and the spacer subsequent to implantation.

Yet another aspect of the present invention is a method. The method may be based upon the surgeon's use of the kit in accordance with the other aspect of the present invention. The method includes providing a fixation device for implant location intermediate the two bone bodies, providing a spacer for implant location intermediate the two bone bodies and adjacent the fixation device, and selecting to implant the device and the spacer together or separately as at least one of the device and the spacer. The method also includes implanting the selected at least one of the device and the spacer at an implant location intermediate the two bone bodies using at least one implantation tool. The at least one tool have a portion for engaging and holding the at least one of the device and the spacer during implantation of the at least one of the device and the spacer and for releasing the held at least one of the device and the spacer subsequent to implantation.

FIG. 12 shows a flow chart with top-level method steps of an example of the method for use with the example controlled subsidence device described herein. Of course, the method will vary dependent upon the type of fixation device. With the example method, the controlled subsidence device is provided at step 202. The spacer is also provided at step 204. At this point it is selected to implant the device and the spacer either together or separately as a selected at least one of the device and the spacer (see step 206). The selected at least one of the device and the spacer is implanted using at least one tool (see step 208).

Turing to more details of the example method, the method is for achieving selective implantation with respect to two bone bodies (e.g., vertebrae 10, 12). The bone bodies (e.g., vertebrae 10, 12) have relative superior and inferior locations. The method includes providing a controlled subsidence device 20 for implant location intermediate the two bone bodies (e.g., vertebrae 10, 12). The device 20 includes first and second lateral portions 118, 120 for location on first and second lateral sides of a chamber to be at least partially bounded by the device 20 and the bone bodies, and at least one interface member 104 extending transverse to a lateral direction and configured to provide controlled subsidence of the at least one interface member into at least one of the bone bodies. The method includes providing a spacer 22 for implant location intermediate the two bone bodies and within the chamber.

The spacer 22 and the device 20 are relatively sized such that the lateral portions 118, 120 of the device substantially bound the spacer in the lateral directions, and such that the spacer does not prevent controlled subsidence of the at least one interface member 104 into the at least one of the bone bodies. The method includes selecting to implant the device 20 and the spacer 22 together or separately as at least one of the device and the spacer. The method includes implanting the selected at least one of the device and the spacer at an implant location intermediate the two bone bodies using at least one implantation tool (e.g., 32 and/or 34). The at least one tool has a portion (e.g., 44 and/or 84) for engaging and holding the at least one of the device and the spacer during implantation of the at least one of the device and the spacer and for releasing the held at least one of the device and the spacer subsequent to implantation.

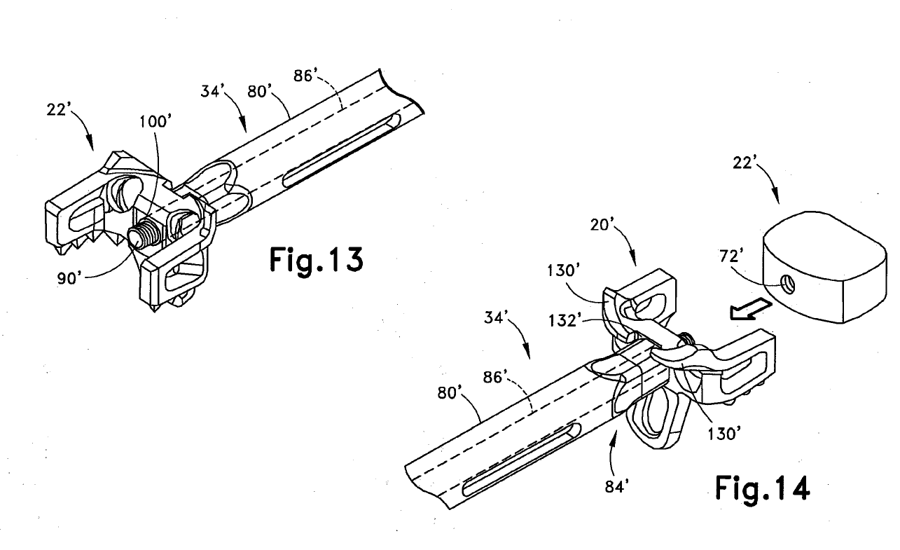

Of course, it is to be appreciated that the specific examples presented need not be specific limitations upon the present invention. Such an understanding has been repeated presented herein. As a further indication that specific examples need not be specific limitations upon the present invention, another example is presented in FIGS. 13 and 14. A tool 34′, a fixation device 20′ and a spacer 20′ are shown. These are at least similar to counterparts that were previously described and thus similar reference numerals are use, but with a “′” (prime) to indicate at least some difference.

Focusing first upon FIG. 13, attention is directed to portion 90′ of the tool 34′ extending through the opening 100′ on the fixation device 22′. First, it should be noted that the portion 90′ is not cone-shaped but instead is threaded. Also, note that the threaded portion 90′ does not threadingly engage the opening 90′ in the fixation device 22. The diameter of the threaded portion 90′ is smaller than the diameter of the opening 100′ such that the threaded portion 90′ may pass through the opening 100′ without interference.

The threaded portion is for engagement with the spacer 22′ (see FIG. 14). As such, the threaded portion 90′ is for engaging and holding the spacer 22′ during implantation. Of course, the spacer 22′ is released subsequent to implantation.

Still with reference to FIG. 14, it can be appreciated that the device 20′ and its projections 130′ are be configured to engage and mate with the static, non-rotating portion of the shaft 80′ of the tool 34′. Such engagement/mating is such that the device 20′ is held against rotation as the rod 86′ with the threaded segment 92′ is rotated within the threaded opening 72′ of the spacer 22′. In the shown example, the shaft 80′ has a generally square cross-section at the engagement end portion 84′ of the shaft. As such in the shown example of the device 20′, the space 132′ between the two projections 130′ is configured (e.g., generally square profile) to receive the square end of the shaft 80′ such that with the square end so received, the device 20′ is held against rotation relative to the shaft of the tool 34′. Thus the rod 86′ can be rotated and the threaded segment 90′ can be rotationally tightened or loosened within the opening 72 in the spacer 22′. The square end of the shaft 80′ may be considered a portion for engaging and holding the device 20′ during implantation and for release subsequent to implantation.

Focusing upon the spacer 22′, it should be noted that the shown example is solid (e.g., no hollow interior). Also, note that the shown example of the spacer 22′ does not have undulations on the superior and inferior faces. Still further it should be noted that the opening 72′ need not be preformed to have internal threads. So long as the material of the spacer 22′ is sufficiently able to grip (e.g., form to and/or be slightly deformed by) the threaded segment 90′, the spacer 22′ can be engaged and held by the threaded segment 90′. Still further, it is to be noted that one specific example of the spacer 22′ is a piece of bone that has been shaped as desired and such is shown within the example of FIG. 14.

With the spacer 22′ engaged and held by the threaded segment 90′ (regardless of material, pre-threading, etc.) the fixation device 20′ is entrapped onto the tool 34′. Specifically, the device 20′ is sandwiched between the spacer 22′ and the square shaped segment of the engagement end portion 84′. Thus, the tool 34′, the spacer 22′ and the device 22 can be moved together. Such is useful if the surgeon wishes to make slight position adjustments during implantation. Specifically, the surgeon has the ability to move the spacer 22′ and the device 20′ back out from location between the two bone bodies.

While embodiments and applications of this invention have been shown and described, it would be apparent to those skilled in the art that many more modifications are possible without departing from the inventive concepts herein. The invention, therefore, is not to be restricted except in the spirit of the appended claims.

What is claimed:

1. A kit for achieving selective implantation with respect to two bone bodies, the kit including:
a fixation device for implant location intermediate the two bone bodies;
a spacer for implant location intermediate the two bone bodies and adjacent the fixation device; and
at least one implantation tool, the at least one tool have a portion for engaging and holding at least one of the device and the spacer during implantation of the at least one of the device and the spacer for implant location intermediate the two bone bodies and for releasing the held at least one of the device and the spacer subsequent to implantation.

2. The kit as set forth in claim 1, wherein the at least one implantation tool includes a portion for engaging and holding the spacer regardless of the spacer being implanted separate from the device or the spacer being implanted together with the device.

3. The kit as set forth in claim 2, wherein the device has an opening, the portion for engaging and holding the spacer extends through the opening in the device to the spacer.

4. The kit as set forth in claim 1, wherein the at least one implantation tool includes first and second tools, the first tool includes a portion for engaging and holding the spacer during implantation of the spacer separate from the device, the second tool includes a portion for engaging and holding the spacer and the device together during implantation of the spacer and device together.

5. The kit as set forth in claim 1, wherein the portion for engaging and holding at least one of the device and the spacer during implantation includes threads that mate with threads on the at least one of the device and the spacer.

6. The kit as set forth in claim 1, wherein the portion for engaging and holding at least one of the device and the spacer during implantation includes a conical segment that wedges into an opening in the at least one of the device and the spacer.

7. The kit as set forth in claim 1, wherein the at least one implantation tool includes a first portion for engaging and holding the device during implantation and a second portion for engaging and holding the spacer during implantation, simultaneous with the implantation of the device, and the first and second portions being configured to release the held device and spacer subsequent to implantation.

8. The kit as set forth in claim 1, wherein the at least one implantation tool includes a portion for engaging and holding the spacer, and the portion extends through a hole in the device to the spacer and is threadingly engagable with the spacer without being threadingly engaged with the hole in the device.

9. The kit as set forth in claim 1, wherein the device is one of a controlled subsidence device and a non-controlled subsidence device.

10. The kit as set forth in claim 1, wherein the bone bodies having relative superior and inferior locations, the device includes first and second lateral portions for location on first and second lateral sides of a chamber to be at least partially bounded by the device and the bone bodies and at least one interface member extending transverse to a lateral direction and configured to provide controlled subsidence of the at least one interface member into at least one of the bone bodies, the spacer being for implant location within the chamber, the spacer and the device being relatively sized such that the lateral portions of the device substantially bound the spacer in the lateral directions and such that the spacer does not prevent controlled subsidence of the at least one interface member into the at least one of the bone bodies.

11. A method for achieving selective implantation with respect to two bone bodies, the method including:
providing a fixation device for implant location intermediate the two bone bodies;
providing a spacer for implant location intermediate the two bone bodies and adjacent the fixation device;
selecting to implant the device and the spacer together or separately as at least one of the device and the spacer; and
implanting the selected at least one of the device and the spacer at an implant location intermediate the two bone bodies using at least one implantation tool, the at least one tool have a portion for engaging and holding the at least one of the device and the spacer during implantation of the at least one of the device and the spacer and for releasing the held at least one of the device and the spacer subsequent to implantation.

12. The method of claim 11, wherein the at least one implantation tool includes first and second tools, the first tool includes a portion for engaging and holding the spacer during implantation of the spacer separate from the device, the second tool includes a portion for engaging and holding the spacer and the device together during implantation of the spacer and device together, the step of implanting the selected at least one of the device and the spacer includes selecting between the first and second tools as an initial use tool depending upon the selection to implant the device and the spacer together or separately.

13. The method of claim 11, wherein the step of selecting between the first and second tools as an initial use tool includes selecting the first tool, and the step of implanting the selected at least one of the device and the spacer includes implanting the spacer separate from the device.

14. The method of claim 11, wherein the step of selecting between the first and second tools as an initial use tool includes selecting the second tool, and the step of implanting the selected at least one of the device and the spacer includes implanting the spacer together with the device.

15. The method of claim 11, wherein the step of selecting between the first and second tools includes selecting a single tool that includes a first portion for engaging and holding the device during implantation and a second portion for engaging and holding the spacer during implantation, simultaneous with the implantation of the device, and the first an second portions being configured to release the held device and spacer subsequent to implantation.

16. The method of claim 11, wherein the bone bodies having relative superior and inferior locations, the device including first and second lateral portions for location on first and second lateral sides of a chamber to be at least partially bounded by the device and the bone bodies and at least one interface member extending transverse to a lateral direction and configured to provide controlled subsidence of the at least one interface member into at least one of the bone bodies, the spacer and the device are relatively sized such that the lateral portions of the device substantially bound the spacer in the lateral directions and such that the spacer does not prevent controlled subsidence of the at least one interface member into the at least one of the bone bodies.

17. An implantation tool including:
a first portion for engaging and holding a fixation device during implantation at a location intermediate two bone bodies; and
a second portion for engaging and holding a spacer during implantation, simultaneous with the implantation of the fixation device, at the location intermediate the two bone bodies and adjacent to the fixation device;
the first and second portions being configured to release the held device and spacer subsequent to implantation.

18. The tool of claim 17, wherein the first portion is threaded.

19. The tool of claim 17, wherein the second portion is one of a cone and threaded.

20. The tool of claim 17, wherein the device has an opening, the second portion has a diameter that is smaller than a diameter of the opening in the device.

21. The tool of claim 17, wherein the device has an opening, the first portion of the tool engages with the opening of the device, and the second portion of the tool extends through the opening in the device and engages the spacer.

22. The tool of claim 17, wherein the first portion is configured for engaging and holding the device which is a controlled subsidence device during the implantation, wherein the device including first and second lateral portions for location on first and second lateral sides of a chamber to be at least partially bounded by the device and the bone bodies, and at least one interface member extending transverse to a lateral direction and configured to provide controlled subsidence of the at least one interface member into at least one of the bone bodies, and the second portion is configured for engaging and holding the spacer with the spacer and the device being relatively sized such that the lateral portions of the device substantially bound the spacer in the lateral directions and such that the spacer does not prevent controlled subsidence of the at least one interface member into the at least one of the bone bodies.
